(19) United States
(12) Patent Application Publication
(10) Pub. No.: US 2025/0345658 A1
(43) Pub. Date: Nov. 13, 2025

(54) ADAPTIVE INTERACTIVE TRAINING ENVIRONMENT

(71) Applicant: Intelligent Training Group ApS, Sorø (DK)

(72) Inventors: Peter MØLLER HANSEN, Sorø (DK); Brian OVERKÆR, Sorø (DK); Anders WILLEMOES HANSEN, Sorø (DK)

(21) Appl. No.: 18/286,172

(22) PCT Filed: Apr. 7, 2022
(86) PCT No.: PCT/EP2022/059287

§ 371 Date: Oct. 9, 2023

(30) Foreign Application Priority Data
Apr. 9, 2021 (EP) 21167675.4

(51) Int. Cl. A63B 24/00; A63B 71/06

(52) U.S. Cl. CPC A63B 24/0075; A63B 71/0622; A63B 2071/0644; A63B 2220/18; A63B 2220/30; A63B 2225/20; A63B 2225/50; A63B 2230/06; A63B 2230/40

(57) ABSTRACT

The present invention relates to a computer-implemented method for providing an interactive training environment for athletic activities performed by a participant on an exercise machine the method comprising the steps of, —receiving intensity data describing the intensity of an athletic activity being performed on said exercise machine; —generating a virtual training world and displaying a visual representation of the virtual training world, the visual representation representing the participant position in the virtual training world—continuously updating the visual representation of the virtual training world and thereby creating the visual impression that said participant is moving into the virtual training world with a speed or inclination which is determined based on the received intensity input. Thereby the virtual training environment adapts to the training intensity of the participant.

### CROSS-REFERENCE TO RELATED APPLICATIONS

This application is a U.S. national stage entry under 35 U.S.C. § 371 of PCT International Patent Application No. PCT/EP2022/059287, filed Apr. 7, 2022, which claims the benefit of and priority to EP patent application Ser. No. 21/167,675.4, filed Apr. 9, 2021, the entire contents of each of which are incorporated herein by reference.

### TECHNICAL FIELD

The present invention generally relates to providing an adaptive interactive training environment for athletic activities performed on an exercise machine.

### BACKGROUND

Athletic activities, such as cycling and running, typically take place outside. Training for such activities often takes place indoors, using exercise machines such as a bike trainer, treadmills or step machines. One popular type of bicycle-based training takes the form of indoor cycling classes where a plurality of participants pedal stationary bikes while being directed by an instructor. The stationary bikes mimic real bikes in that they include pedals, a seat, and handle bars, and further include some type of adjustable resistance mechanism to provide variable resistance levels. The instructor typically coaches the class through a workout plan having a plurality of sequential intervals. During each interval, the class will be instructed to use a particular exercise position (seated or one or more standing positions) at a particular pace (an RPM or cadence) and at a resistance level. The resistance level may be a specific resistance level or a resistance level that the participant perceives as corresponding to the instructor's instructions. For example, the instructor may ask the class to pedal for one minute at an RPM of 70 to 80 with medium-high resistance. The precise resistance level used by each participant is typically chosen by the participant and may vary widely from participant to participant, depending on the individual participant's fixed level and how they are feeling.

While in-person cycling classes are very popular, distributed and/or virtual training classes are becoming increasingly popular. In one example, an instructor is in a first location while one or more participants are located at one or more remote locations. The instructor communicates with the students using video and audio, such as through an internet webpage. Alternatively, dedicated software may be used to provide communication between the instructor and participants. The class proceeds similarly to an in-person class except that the participants and the instructor are remote from one another. U.S. Pat. No. 9,174,085 to Foley et al. discloses an example of a method for providing cycling classes to remote participants. Similar training classes are available for runners using treadmills either for in-person classes or remote training.

Another popular approach to indoor training is the provision of virtual training environments. One example is Zwift™ which provides a virtual “world” in which participants may cycle or run. Participants use an indoor bike or treadmill to participate. Many participants use a cycle trainer, which is a device that attaches to the rear wheel or drivetrain of a bicycle and provides variable resistance levels to a participant. Software provides for two-way communication between the virtual “world” and the exercise equipment. The participant's speed, cadence, power output and other factors may be communicated to a remote server and the participant's position within the virtual “world” is adjusted to provide semi-realistic correspondence between the participant's effort and movement in the world. Depending on the equipment being used by the participant, the remote server may also communicate control signals to the exercise device that causes the exercise device to adjust resistance. The communication protocol may be Bluetooth or Ant+. For example, when the participant comes to a virtual incline in the virtual world, the server may communicate to the exercise equipment to increase the resistance level. Alternatively, the participant may manually adjust the resistance level of their equipment to coordinate their effort with their apparent position in the virtual world. In this example, the virtual world allows for a very large number of participants to “ride” or “run” in the same virtual world and to perceive avatars of each other representing their relative positions and speeds. Communication between remote participants is also allowed. However, the virtual world is fixed, in the sense that the path or road to be traveled by the avatar is the same each time the participant visits the virtual world. A large number of possible roads and paths may be provided, but each path or road is the same, visit to visit. Features within the virtual world vary during exercise sessions and between sessions, such as the weather, sounds, and the apparent position of the sun. Certain virtual training systems also allow for training plans wherein the resistance level experienced by the participant is predefined so as to provide a specified sequence of intervals. For example, a participant who is training for speed may make use of a training plan including a plurality of high speed “sprints” separated by recovery periods. A participant who is training for climbing may use a plan having a plurality of high effort, low cadence intervals separated by recovery periods.

The above-described solutions are all defined by having a predefined training plan and the participant is then supposed to train according to that plan and the virtual training environment defines/dictates at least part of the training. The participant might be interested in training more freely where a training plan does not dictate the effort to be made by the participant and where the participant has the possibility of controlling the training and still use a virtual training environment.

### SUMMARY OF THE INVENTION

This is obtained by a computer-implemented method for providing an interactive training environment for athletic activities performed by a participant on an exercise machine, the method comprising the steps of,
receiving intensity data describing the intensity of an athletic activity being performed on said exercise machine;
generating a virtual training world and displaying a visual representation of the virtual training world, the visual representation representing the participant position in the virtual training world
continuously updating the visual representation of the virtual training world and thereby creating the visual impression that said participant is moving into the virtual training world with a speed or inclination which is determined based on the received intensity input.

Thereby the virtual training environment adapts to the training intensity of the participant, if e.g. the participant starts training with a faster speed e.g. higher RPM on a cycle or faster steps on a treadmill then then the participant will appear to move faster into the virtual training. Similarly, if the participant slows down e.g. lowers the RPM on a training cycle or takes slower steps on a treadmill then then the participant will appear to move slower into the virtual training world. Further when moving through the virtual training world it is also possible to update the visual impression to indicate inclination whereby instead of creating the visual impression of just moving straight through the virtual training world the impression can be that the participant is moving through with an upwards inclination. This inclination is also determined based on the intensity input and if the RPM on a training cycle is lowered then this could also change the inclination to a higher upwards inclination. It could, however, also be another intensity parameter that influences the inclination namely an input describing the resistance on the training cycle or the actual inclination on a treadmill. If the resistance on the training cycle or the inclination of the treadmill is increased then this is visualized by a more upwards inclination when moving by the participant.

The virtual training world could be a cartoon like underwater world, a world in space or any other world based on the creativity and fantasy of the virtual world designer. It is all about designing a world that enables illustration of speed and inclination and at the same time motivates the participant. Another example is that the virtual world is a known world from cartoons, movies, computer games or the like, where elements being passed by the participant during the exercise correspond to elements and environments which can be recognized from these well-known worlds. As an alternative to the fictive environments the world could also be completely or at least partially based on actual real world data e.g. from google maps or similar. Thereby the inclination and speed adapts to the intensity data and the inclination of the surface from the real world would adapt to the intensity. A result would be that Alpe d'Huez could be illustrated as flat if the participant has a low intensity when passing the area being a mountain in the real world, or when passing a real world flat area with a high intensity it could be presented with an inclined surface. The participant might experience passing houses from the home city of the participant, but where inclination adapts to the intensity, maybe the home of the participant is shown laying near a steep surface, which is different from the real world position of the home.

In an embodiment the intensity input comprises intensity data measured on the exercise machine. Such intensity data depends on the type of exercise machine which could be a spinning cycle/bike trainer, treadmill, step machine, cross trainer, elliptical trainer, rowing machine, ski trainer, virtual reality equipment (e.g. combined with augmented reality) or the like. Based on this, the intensity data could be measured on the exercise machines and e.g. be inclination/elevation of a training device, Pulse/Heart Rate measured via handles, Watt, FTP, % FTP, Position, Speed, Distance Traveled, MAP, % MAP, FTHR, % FTHR, Power Zones, LTHR, % LTHR, VO2max, % VO2max, RPE Levels, Kcal, Strokes, Time (time will affect the RPE levels) or the like.

In a specific embodiment intensity data measured on the exercise machine comprises data such as exercise speed or exercise resistance.

In an embodiment intensity input comprises intensity data measured on the participant and more specifically such data comprises physiological data such as heart rate and oxygen consumption.

In an embodiment said intensity data is received by a computer performing said computer implemented method via wireless communication means.

In an embodiment intensity data being exercise speed determines the speed in which the participant is moving into the virtual training world and the exercise resistance determines the inclination in which the participant is moving into the virtual training world. Thereby the participant receives feedback directly relating to the performance.

In an embodiment any change in intensity data results in a corresponding change in at least speed or inclination in which the participant moves into the virtual training world. Thereby the participant immediately experiences feedback in the visual training world mirroring the intensity.

In an embodiment predefined intervals of intensity are each linked to a specific speed or inclination with which the participant moves into the virtual training world. Thereby the visual training world does not have to change each time a small intensity change happens, and this would require fewer computing resources.

In an embodiment the virtual training world illustrates a training surface on which the participant moves into the virtual training world. Illustrating the surface is an advantage for a realistic feel of inclination and speed change in the visual representation of the training world.

In an embodiment the training surface is at least partially color-coded or pattern-coded to match the corresponding specific speed or inclination. This can enhance the speed experience.

In an embodiment, the method—besides from representing a visual representation of the virtual training world—also provides visually represented feedback indicia including at least one of the following: training time, time in interval, time remaining in workout, target intensity, exercise position, resistance level of the exercise device, actual participant RPM, actual participant heart rate, and actual participant wattage. This can aid in increasing the feedback realism to the participant according to the actual intensity.

In an embodiment the method interprets said intensity data and associates it with a plurality of virtual training world features. This can be a supplement or alternative to creating a visual representation of intensity.

The invention further relates to a system for providing an interactive training environment for athletic activities performed by a participant on an exercise machine and a machine readable storage medium containing one or more programs for performing a method of providing an interactive training environment for athletic activities according to the above.

### SUMMARY OF FIGURES

FIG. 1 illustrates a training system comprising a participant on an exercise machine being a bike trainer viewing a display displaying a visual representation of an interactive training environment in accordance with an embodiment of the present invention.

FIG. 2 illustrates a visual representation of a virtual training world according to the present invention wherein the participant is moving into the virtual training world with a first inclination,

FIGS. 3a-3f illustrate a training session using a training system according to the present invention,

FIG. 4 is a flowchart of a method according to the present invention.

FIG. 5 is a schematic view representing a system in accordance with an embodiment of the present invention.

### DETAILED DESCRIPTION

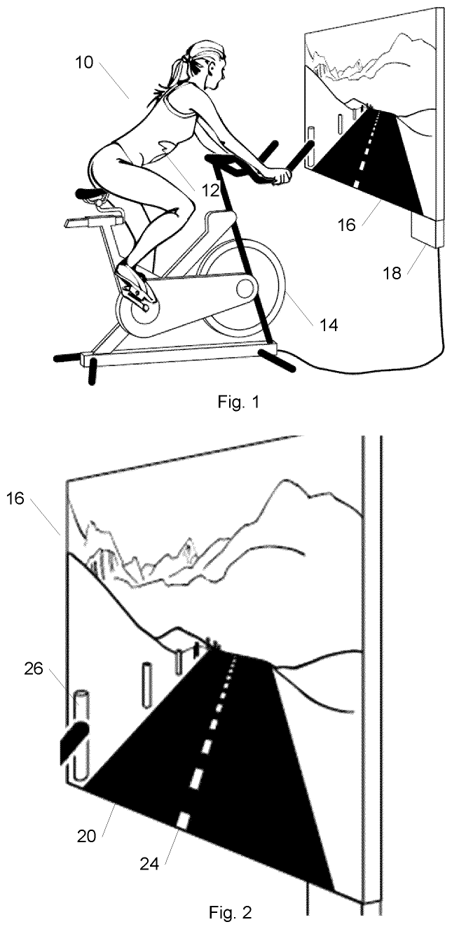

FIG. 1 illustrates a training system 10. A participant 12 on an exercise device and in this example the exercise device is a bike trainer or stationary bike 14 that is designed to simulate riding a bicycle, including providing resistance to pedaling. A display 16 displays a visual representation of a virtual training world. In this example, the display is a wall-mounted television screen, but it could also take the form of a display of a telephone, tablet, or computer, a built-in display on the stationary bike 14 or any other form allowing the participant to view the visual representation. A computer 18 is shown attached to display 16. In this example, the computer generates the visual representation of the virtual training world and has a wired connection to the exercise device so as to provide one-way or two-way communication between the exercise device and the computer 18. The computer may take a wide variety of forms, including a stand-alone computer, a phone, a tablet, a laptop, a TV control box (i.e., Apple TV), a computer-integrated into a smart display, or any other type of computing device capable of providing the visual representation to the display. The connection to the display and/or the exercise device may be wired or wireless.

Referring now to FIG. 2, the visual representation of the virtual training world will be described in more detail. The visual representation is provided on display 16. In this example a virtual training surface 20 is illustrated in the foreground of the visual representation such that the participant sees the virtual training surface as being traveled on or along and this surface aids the visual impression that the participant is moving into the virtual direction with a speed and inclination. Alternatively, there might not be an actual surface and other elements in the virtual representation aids to create the moving impression of speed and inclination. The virtual training surface could have borders on one or both sides and/or a center line 24 as illustrated in this example and have the appearance of a road with a center line. A border could have the appearance of alternating black and white sections. This may be pattern-coded to the corresponding intensity to indicate the intensity of the training performed by the participant and further this is also useful to give the impression of speed (e.g. when running faster or when RPM on the training cycle increases) and the participant gets feedback accordingly from the visual representation.

Moving from the foreground of the visual representation to the background in this illustrated example, along the virtual training surface 20 poles 26 are positioned and such poles being passed is one way of creating the visual impression that the participant is moving through the virtual training world by making the visual impression that the poles move towards the participant. In the illustration, an example is also shown on how the visual representation can provide the impression that the participant is on a surface that is flat with no inclination. In general, techniques on how to visually represent respectively speed and inclination is done by using well-known drawing techniques using surroundings, relative positions of objects, surfaces, etc. to create impressions.

Typically, an exercise device for use with the system does not provide for steering or turns, but instead the visual representation will be updated to show the participant moving into the virtual training world as following the curves. As discussed previously, world features such as turns may be pseudo-randomly generated such that the path of the virtual training surface may be different every time a participant uses the system. Therefore, mainly the speed and inclination that the participant is moving into the virtual training world is determined by the training of the participant e.g. based on intensity feedback from the exercise device, the participant or a combination. Further world features are represented in the background of FIG. 2, including mountains and hills. In a specific embodiment the participant training on the exercise device could be wearing VR glasses or similar and in such a setup, turns could be generated based on input from the VR glasses, e.g. by generating a turn corresponding to the side that the VR glasses are directed towards. In such a setup speed or inclination is determined based on the received intensity input from the exercise device, but the direction of turns is based on input from the VR glasses.

The visual impression of speed and inclination and how the participant moves into the visual training world changes based on a detected intensity change, e.g. when a detection of intensity changes more than 5% then the inclination is changed with 5%, thereby as training intensity changes then the visual representation changes accordingly to visualize the intensity. Of course, any change in intensity by the participant could influence the visual impression of speed and inclination and how the participant moves into visual training world. To save computer power it can be an advantage to only alter this in intervals.

As already mentioned, some of the parameters used in the visual representation of the virtual training world to visualize the intensity are the inclination in the direction that the participant is moving through the virtual training world and the speed, where the speed and/or the inclination e.g. will increase as the intensity increases. Alternative ways of illustrating an intensity change could be changing the appearance of objects being passed or maybe even adding objects based on the detected intensity. In general, the participant needs to get a visual feedback from the visual representation that intensity is changing. The visual representation can either be a natural environment as illustrated or a fictive world.

According to the present invention the training intensity of the participant dictates the appearance of the visual training world. The training intensity could be measured in real time by measuring the intensity of an athletic activity performed on the exercise device as an intensity input. The inclination of the training surface and or moving speed of the training surface is then a function of the measured training intensity and is changed based on the intensity input. The intensity input could be measured by measuring the pulse of the participant, the power from the participant to the exercise device (such as cadence), RPM, resistance settings on the exercise device, etc.

When the inclination of the training surface changes as a function of the intensity input then in one embodiment the inclination could change as a linear function of the intensity level where any change of intensity would result in a change in inclination and/or speed. As an alternative the intensity input could also be grouped in intervals where each interval would result in a training surface having one inclination. If e.g., the intensity input is measures as RPM then 50-60 RPM could result in the training surface having one inclination, whereas 60-70 RPM results in another inclination being less steep. An example of the relation between intensity level and inclination and/or moving speed of the training surface is shown in the below table.

| Intensity level | Inclination of training surface | Speed of training surface |
|---|---|---|
| High | High climbing | Very slow |
| Medium | Medium climbing | Slow |
| Low | No Inclination straight | Normal |
| No Intensity | Medium descending | Fast |
| No intensity | High descending | Very fast |

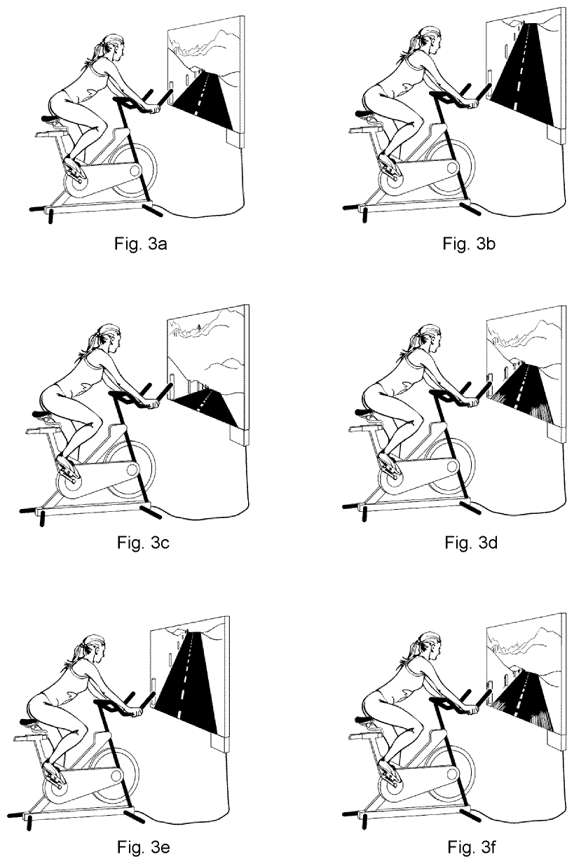

FIGS. 3a-3f illustrate a training session using a training system according to the present invention.

In FIG. 3a the participant is training with a relatively low intensity e.g. where both the resistance of the training cycle as well as the speed of training (RPM) is low. The visual representation is illustrated as a straight surface and the participant appears to move into the virtual training world with low speed and no inclination.

In FIG. 3b the participant is training with a high intensity e.g. where the resistance of the training cycle is high and the speed of training (RPM) is low. The visual representation is illustrated as an inclined surface with a relatively high upwards inclination and the participant appears to move into the virtual training world with low speed and high upwards inclination.

In FIG. 3c the participant is training with a high intensity e.g. where the resistance of the training cycle is low and the speed of training (RPM) is high. The visual representation is illustrated as an inclined surface with a relatively low downwards inclination and the participant appears to move into the virtual training world with high speed and low downwards inclination.

In FIG. 3d the participant is training with a high intensity e.g. where the resistance of the training cycle is higher than in 3c and the speed of training (RPM) is also higher. The visual representation is illustrated as a straight surface with no inclination and the participant appears to move into the virtual training world with high speed and no inclination.

In FIG. 3e the participant is again training with a high intensity e.g. where the resistance of the training cycle is high and the speed of training (RPM) is low. The visual representation is again illustrated as an inclined surface with a relatively high upwards inclination and the participant appears to move into the virtual training world with low speed and high upwards inclination.

Finally in FIG. 3f the participant is still training with a high intensity e.g. here the resistance of the training cycle is lower than in 3e, but the speed of training (RPM) is much higher. The visual representation is illustrated as a straight surface with no inclination and the participant appears to move into the virtual training world with high speed and no inclination.

The above examples are given where the intensity data is a direct measurement of resistance and RPM on the cycle. The participant appears to move into the virtual training world with a speed and inclination which is determined based on the received intensity input. If e.g. only RPM is measured (blue column below) then the relation between speed and inclination with which the participant moves through the virtual training world could e.g. be as follows:

| | Level of inclination | |
|---|---|---|
| RPM | (factors) | Speed/movement (factors) |
| 50-60 | 7 | A product of (a) RPM and (b) Level of |
| | | inclination factor 7 |
| 60-70 | 6 | A product of (a) RPM and (b) Level of |
| | | inclination factor 6 |
| 70-80 | 5 | A product of (a) RPM and (b) Level of |
| | | inclination factor 5 |
| 80-90 | 4 | A product of (a) RPM and (b) Level of |
| | | inclination factor 4 |
| 90-100 | 3 | A product of (a) RPM and (b) Level of |
| | | inclination factor 3 |
| 100-110 | 2 | A product of (a) RPM and (b) Level of |
| | | inclination factor 2 |
| 110+ | 1 | A product of (a) RPM and (b) Level of |
| | | inclination factor 1 |

As can be read from the above as the RPM increases the inclination falls and the speed is determined based on a combination of the illustrated inclination and the RPM.

If e.g. both the RPM is measured as well as a resistance setting is part of the measured intensity data, then both the speed and the inclination with which the participant moves through the virtual training world is determined based on a combination of the illustrated inclination and the RPM.

The intensity data is in the above examples measured as RPM and Resistance on bike, but the intensity data could also one or more of the following:

Inclination/elevation of the training device, Pulse/Heart Rate, Watt, FTP, % FTP, Position, Speed, Distance Traveled, MAP, % MAP, FTHR, % FTHR, Power Zones, LTHR % LTHR, VO2max, % VO2max, RPE Levels, Kcal, Strokes, Time (time will affect the RPE levels), Colors, Moves, Points or the like.

Further, the above examples have been given where the training device is a spinning cycle/bike trainer. The training device/exercise machine could of cause be any type of device including a treadmill, step machine, cross trainer, elliptical trainer, rowing machine, ski trainer, virtual reality equipment (e.g. combined with augmented reality).

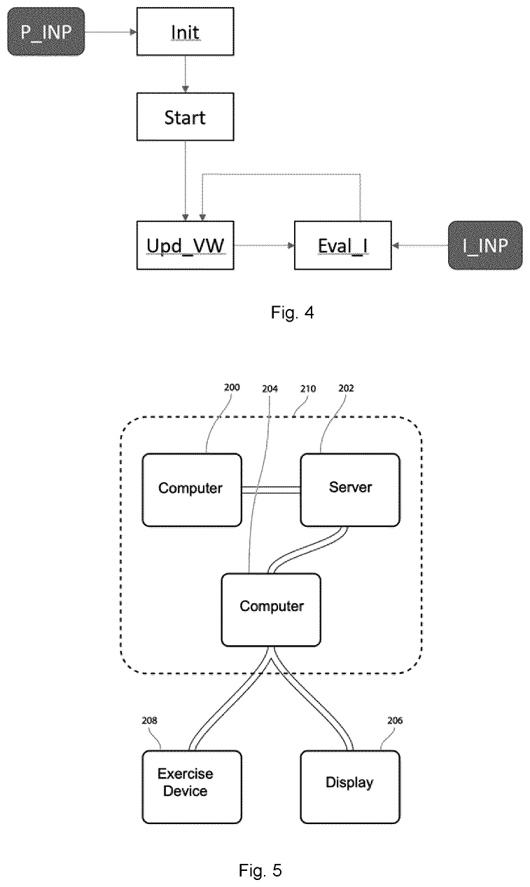

Referring now to FIG. 4, steps of a method in accordance with the present invention is described. In the initialization step (Init) a participant selects via participant input (P_INP) a type of training world, such as “under water.” “outer space,” or “on land.” This choice may then determine some of the visual features of the virtual training world. The possible training worlds may be preinstalled on the system, but it may also be possible to add further worlds or features to the system e.g., by downloading it via an internet connection. The participant may also specify music and this music may be adjusted during the training to suit the received intensity data, such as by adjusting the beat of the music.

Then the participant may start the workout session (Start) and a virtual training world is generated. Generation of a virtual training world includes pseudo-randomly generating a plurality of world features, which may include turns in the training surface and features of the turns. In some examples, the system uses multiple layers to generate all visuals that are all governed by the generation algorithm corresponding to the algorithm already described above. The system could pick from a pool of different background landscapes, set dressings, roads and sky boxes to match the difficulty of a workout. Additionally, there may be characters such as animals or flying spaceships that each have their own generation algorithms and path finding Artificial Intelligence (AI) protocols. A visual effects layer may add more depth to the image. As an example, elements such as water drops or fog are rendered on a camera layer that overlays all other content. Certain embodiments of the present invention use a proprietary algorithm to create a visual representation of a parametric workout. The JSON input, consisting purely of numbers over time, is taken and interpreted by an “AI system”. This system is able to connect the parametric values in the given workout source to virtual assets (3D meshes of environment and road) and thus create a visual representation. In order to ensure that the same workout source will always deliver different worlds (to keep the participant excited even when re-visiting workouts) a random seed consisting of five numbers is used. This leads to 99999×99999 possibilities in combination for each individual world that is available and essentially endless, non-repetitive worlds for the participant to experience.

The AI protocols could construct the layers such that elements in the individual layers do not appear to interfere with each other. As an example, the training surface has turns. The path of the training surface may constrict where a layer “behind” the training surface has objects that would otherwise interfere with the training surface.

In an example, each “layer” has a set of objects to randomly choose from. The objects may be placed pseudo-randomly, but the positioning is conditioned on layers that appear in the front. The layer at the back may be the horizon, i.e. a background color. If mountains are present, the second layer may be mountains for which the position, size and other features may be pseudo-randomly generated. There may also be a layer with smaller objects such as tree, plants, animals etc. The virtual training surface is a further layer.

Thereby the type of training world is generated, and the variable is the speed and inclination with which the participant should move through this generated world. Alternative or additional ways of illustrating could be by adding objects based on the detected intensity or maybe even changing of objects.

In the next step (Upd_VW) the image is updated according to an initial intensity with an initial speed and inclination, the impression of speed can e.g. be made by updating the image to create a visual impression that elements in the virtual world are being passed at a specific speed and similarly the impression of inclination is obtained by a visual impression that elements in the virtual world are being passed at specific angle. When a training surface is illustrated, this surface can be an important tool for creating visual impression of speed and inclination.

After updating the virtual training world, the intensity of the training is evaluated based on the received intensity data (I_INP) and based on these data it is decided if the speed and/or inclination should be changed when performing the next update of the image (Upd_VW).

When starting the training, the participant could move through the virtual training world with a slow speed and no inclination based on specific settings identifying speed and inclination which could be set e.g. when initializing the workout session, then only based on changes in the intensity data relative to the initial speed and inclination the speed or inclination by which the participant moves through the virtual training world will change.

In one embodiment changes to speed/or inclination could occur immediately as the intensity data changes or changes could only occur in intervals. The inclination may go up when the RPM goes down. The same may be the case with the intensity. The position of the participant on the bike may add or subtract to the inclination. For example, a standing position may add to the inclination. Further the background may move at a speed defined by the intensity input from the training session. Therefore, in the visual representation, when one mountain moves out of the picture bit by bit a new mountain starts to come into the picture.

In an embodiment when starting training, the participant is initially not moving through the virtual training world and then when the participant starts training the inclination and speed is updated according to the intensity.

FIG. 5 provides a schematic view of a system for practicing embodiments of the present invention. A first computer 200 is shown and may be used for the step of selecting or generating a training world. Such a computer may be a stand-alone computer, a laptop, a phone, a tablet or other computing device. The selecting or generating of a virtual training world may take place on this computer, or the computer 200 may be in communication with a server 202. For example, the software associated with the method may run on a server or servers and be accessed by a computer 200 via a webpage. A computer 204 is connected to a display 206 for displaying the visual representation of the virtual training world. The computer 204 may also connect to the exercise equipment 208 for embodiments of the present invention where information is provided between the exercise device and the system. Any of the connections between the various computers and other equipment may be wired or wireless. Box 210 indicates that one or more of the computers 200, 202 and 204 may be a single device performing more than one of the method steps. For example, all computing functions may take place on the server with the display merely displaying what information is provided for the server. However, typically a computer 204 will control the display and may take the form of a computer, laptop, phone, tablet, smart TV, or control box for a TV. The exercise equipment 208 may take a variety of forms, including a stationary exercise bike, such as illustrated at 14 in FIG. 1, a bike trainer, or a treadmill. A biker trainer is a device that connects to the rear wheel or drivetrain of an actual bicycle and provides resistance for the participant. The computer 204 may receive input identifying the resistance of the bike trainer, exercise bike or treadmill, with the treadmill resistance typically being in the inclination of the treadmill belt. Alternatively, the display 206 may provide information to the participant indicating a current resistance or intensity of the current exercise, with the participant manually adjusting the exercise equipment. The display 206 may take a variety of forms, including a computer display, TV, tablet, phone, headset or a display that forms part of the exercise bike or treadmill.

1. A computer-implemented method for providing an interactive training environment for athletic activities performed by a participant on an exercise machine the method comprising the steps of,
receiving intensity data describing the intensity of an athletic activity being performed on said exercise machine;
using a computer for generating a virtual training world and displaying a visual representation of the virtual training world, the visual representation representing the participant position in the virtual training world
based on said received intensity input, said computer generated virtual training world is continuously updated creating the visual impression that said participant is moving into the virtual training world with a speed or inclination corresponding to the received intensity input.

2. The method according to claim 1, wherein intensity input comprises intensity data measured on the exercise machine.

3. A method according to claim 2, wherein intensity data measured on the exercise machine comprises data such as exercise speed or exercise resistance.

4. A method according to claim 1, wherein intensity input comprises intensity data measured on the participant.

5. A method according to claim 4, wherein the intensity data measured on the participant comprises physiological data such as heart rate and oxygen consumption.

6. A method according to claim 1, wherein said intensity data is received by a computer performing said computer implemented method via wireless communication means.

7. A method according to claim 1, wherein a combination of intensity data being exercise speed and exercise resistance determines the speed at which the participant is moving into the virtual training world and the inclination in which the participant is moving into the virtual training world.

8. A method according to claim 1, wherein any change in intensity data result in a corresponding change in at least intensity or inclination in which the participant moves into the virtual training world.

9. A method according to claim 1, wherein predefined intervals of intensity are each linked to a specific speed or inclination with which the participant moves into the virtual training world.

10. A method according to claim 1, wherein the virtual training world illustrates a training surface on which the participant moves into the virtual training world.

11. A method according to claim 9, wherein the training surface is at least partially color-coded or pattern-coded to match the corresponding specific speed or inclination.

12. A method according to claim 1, wherein the method besides representing a visual representation of the virtual training world also visually represents feedback indicia including at least one of total training time, time in interval, time remaining in workout, target intensity, exercise position, resistance level of the exercise device, actual participant RPM, actual participant heart rate, and actual participant wattage.

13. A method according to claim 1, wherein the method further interprets said intensity data and associate it with a plurality of virtual training world features.

14. A system for providing an interactive training environment for athletic activities performed by a participant on an exercise machine, such as a bike trainer, treadmill or a step machine, the system comprising:
a memory; and
at least one processor, coupled to the memory, operative to perform the steps of:
receiving intensity data describing the intensity of an athletic activity being performed on said exercise machine;
generating a virtual training world and displaying a visual representation of the virtual training world, the visual representation representing the participant position in the virtual training world
based on said received intensity input, said generated virtual training world is continuously updated creating the visual impression that said participant is moving into the virtual training world with a speed and inclination which is corresponding to the received intensity input.

15. A machine readable storage medium containing one or more programs for performing a method of providing an interactive training environment for athletic activities according to claim 1.
